(19) United States
(12) Patent Application Publication
(10) Pub. No.: US 2007/0092899 A1
(43) Pub. Date: Apr. 26, 2007

(54) Multiple displacement amplification with blocker DNA

(75) Inventors: Christine A. Hara, Livermore, CA (US); Allen T. Christian, Madison, WI (US)

Correspondence Address: Eddie E. Scott, P.O. Box 808, L-703, Livermore, CA, 94551, US

(73) Assignee: The Regents of the University of California

(21) Appl. No.: 11/509,996

(22) Filed: Aug. 24, 2006

Related U.S. Application Data
(60) Provisional application No. 60/729,269, filed on Oct. 21, 2005.

(51) Int. Cl. C12Q 1/68; C12P 19/34

(52) U.S. Cl. 435/6; 435/91.2

(57) ABSTRACT

A method of whole genome amplification of a nucleic acid sample comprising providing a starting template of the nucleic acid sample, bringing the template together with blocking DNA, bringing the template together with DNA polymerase, and multiple displacement amplification of the template. The method can be used with a starting template of less than 1 ng of the nucleic acid sample.

### CROSS-REFERENCE TO RELATED APPLICATIONS

This application claims the benefit of U.S. Provisional Patent Application No. 60/729,269 filed Oct. 21, 2005 and titled “Multiple Displacement Amplification with Blocker DNA.” U.S. Provisional Patent Application No. 60/729,269 filed Oct. 21, 2005 and titled “Multiple Displacement Amplification with Blocker DNA” is incorporated herein by this reference.

### MULTIPLE DISPLACEMENT AMPLIFICATION WITH BLOCKER DNA

The United States Government has rights in this invention pursuant to Contract No. W-7405-ENG-48 between the United States Department of Energy and the University of California for the operation of Lawrence Livermore National Laboratory.

### BACKGROUND

### FIELD OF ENDEAVOR

The present invention relates to whole genome amplification and more particularly to multiple displacement amplification with blocker DNA.

### STATE OF TECHNOLOGY

United States Patent Application No. 2004/0209298 by Emmanuel Kamberov et al for amplification and analysis of whole genome and whole transcriptome, published Oct. 21, 2004, provides the following state of technology information: “For genomic studies, the quality and quantity of DNA samples is crucial. High-throughput genetic analysis requires large amounts of template for testing. However, the amount of DNA extracted from individual patient samples, for example, is limited. DNA sample size also limits forensic and paleobiology work. Thus, there has been a concerted effort in developing methods to amplify the entire genome. The goal of whole genome amplification (WGA) is to supply a sufficient amount of genomic sequence for a variety of procedures, as well as long-term storage for future work and archiving of patient samples. There is a clear need to amplify entire genomes in an automatable, robust, representative fashion. Whole genome amplification has historically been accomplished using one of three techniques: polymerase chain reaction (PCR), strand displacement, or cell immortalization.”

U.S. Pat. No. 6,977,148 for multiple displacement amplification issued Dec. 20, 2005 to Frank B. Dean et al provides the following state of technology information:

“A number of methods have been developed for exponential amplification of nucleic acids. These include the polymerase chain reaction (PCR), ligase chain reaction (LCR), self-sustained sequence replication (3SR), nucleic acid sequence based amplification (NASBA), strand displacement amplification (SDA), and amplification with Qβ replicase (Birkenmeyer and Mushahwar, J. Virological Methods, 35:117-126 (1991); Landegren, Trends Genetics 9:199-202 (1993)).

Fundamental to most genetic analysis is availability of genomic DNA of adequate quality and quantity. Since DNA yield from human samples is frequently limiting, much effort has been invested in general methods for propagating and archiving genomic DNA. Methods include the creation of EBV-transformed cell lines or whole genome amplification (WGA) by random or degenerate oligonucleotide-primed PCR. Whole genome PCR, a variant of PCR amplification, involves the use of random or partially random primers to amplify the entire genome of an organism in the same PCR reaction. This technique relies on having a sufficient number of primers of random or partially random sequence such that pairs of primers will hybridize throughout the genomic DNA at moderate intervals. Replication initiated at the primers can then result in replicated strands overlapping sites where another primer can hybridize. By subjecting the genomic sample to multiple amplification cycles, the genomic sequences will be amplified. Whole genome PCR has the same disadvantages as other forms of PCR. However, WGA methods suffer from high cost or insufficient coverage and inadequate average DNA size (Telenius et al., Genomics. 13:718-725 (1992); Cheung and Nelson, Proc Natl Acad Sci USA. 93:14676-14679 (1996); Zhang et al., Proc Natl Acad Sci USA. 89:5847-5851 (1992)).

Another field in which amplification is relevant is RNA expression profiling, where the objective is to determine the relative concentration of many different molecular species of RNA in a biological sample. Some of the RNAs of interest are present in relatively low concentrations, and it is desirable to amplify them prior to analysis. It is not possible to use the polymerase chain reaction to amplify them because the mRNA mixture is complex, typically consisting of 5,000 to 20,000 different molecular species. The polymerase chain reaction has the disadvantage that different molecular species will be amplified at different rates, distorting the relative concentrations of mRNAs.

Some procedures have been described that permit moderate amplification of all RNAs in a sample simultaneously. For example, in Lockhart et al., Nature Biotechnology 14:1675-1680 (1996), double-stranded cDNA was synthesized in such a manner that a strong RNA polymerase promoter was incorporated at the end of each cDNA. This promoter sequence was then used to transcribe the cDNAs, generating approximately 100 to 150 RNA copies for each cDNA molecule. This weak amplification system allowed RNA profiling of biological samples that contained a minimum of 100,000 cells. However, there is a need for a more powerful amplification method that would permit the profiling analysis of samples containing a very small number of cells.

Another form of nucleic acid amplification, involving strand displacement, has been described in U.S. Pat. No. 6,124,120 to Lizardi. In one form of the method, two sets of primers are used that are complementary to opposite strands of nucleotide sequences flanking a target sequence. Amplification proceeds by replication initiated at each primer and continuing through the target nucleic acid sequence, with the growing strands encountering and displacing previously replicated strands. In another form of the method a random set of primers is used to randomly prime a sample of genomic nucleic acid. The primers in the set are collectively, and randomly, complementary to nucleic acid sequences distributed throughout nucleic acid in the sample. Amplification proceeds by replication initiating at each primer and continuing so that the growing strands encounter and displace adjacent replicated strands. In another form of the method concatenated DNA is amplified by strand displacement synthesis with either a random set of primers or primers complementary to linker sequences between the concatenated DNA. Synthesis proceeds from the linkers, through a section of the concatenated DNA to the next linker, and continues beyond, with the growing strands encountering and displacing previously replicated strands.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Whole genome amplification (WGA) of DNA can currently be performed by utilizing kits, which contain phi29 polymerase and random priming hexamers. phi29 polymerase is highly processive as well as accurate with its 3′-5′ Exonuclease activity making it an ideal candidate for whole genome amplification. Using it as a method for WGA is called Multiple Displacement Amplification (MDA) due to the displacement of newly replicated strands of DNA by the polymerase as it replicates complimentary strands. Multiple Displacement Amplification (MDA) is described in U.S. Pat. No. 6,977,148 for multiple displacement amplification issued Dec. 20, 2005 to Frank B. Dean et al. U.S. Pat. No. 6,977,148 for multiple displacement amplification issued Dec. 20, 2005 to Frank B. Dean et al is incorporated herein by reference. Kits for Whole Genome Amplification, including REPLI-g Mini and Midi Kits for highly uniform whole genome amplification from small or precious samples, are available from Qiagen Inc.—USA, 27220 Turnberry Lane, Suite 200, Valencia, Calif. 91355.

Although effective when used with 1 ng of starting template or above, these kits are hindered by the amount of starting template needed to replicate multiple copies of DNA. The lower limit of templates makes it virtually impossible to amplify and sequence the genome of single cells as well as DNA templates less than 1 ng. A small quantity of template can also hinder the MDA reaction based on the template to reaction volume ratio. When a template is small in comparison to the reaction volume it is possible for the template to be passed over by the reagents.

Whole Genome Amplification (WGA) using phi29 polymerase and random priming hexamers (known as Multiple Displacement Amplification, MDA) is able to increase starting template mass as much as 100,000 fold with an average sequence length of 12 kb and a nucleotide placement error rate less than 10⁻⁶ making it highly unbiased. However, when the starting mass is too small the lack of complete genome and or amount of DNA can remain the limiting factor in post-amplification analysis.

The ratio of DNA to reaction volume, as well as the template potentially adhering to non-biological surfaces, such as the reaction tube during incubation, may make the template go unnoticed by the reaction reagents. In order to circumvent this problem, Applicants examined the use of carrier DNA and crowding agents in MDA reactions. Researchers have previously examined the use of glycerol and polyethylene glycol as macromolecular crowding agents in order to increase the efficiency of ligation reactions and to decrease background noise in southern hybridizations. However, Applicants tests with glycerol indicated that the reaction was inhibited by the addition.

The present invention provides a method of whole genome amplification of a nucleic acid sample comprising providing a starting template of the nucleic acid sample, bringing the template together with blocking DNA, bringing the template together with DNA polymerase, and multiple displacement amplification of the template. The method of the present invention can be used with a starting template of less than 1 ng of the nucleic acid sample. In one embodiment the method is adapted to amplify nucleic acid of a specific species of organism and the step of bringing the template together with blocking DNA comprises bringing the template together with blocking DNA from a species other than the specific species of organism. In another embodiment, the method is adapted to amplify nucleic acid of a human species and the step of bringing the template together with blocking DNA comprises bringing the template together with blocking DNA from a species other than the human specific species.

The present invention has use by researchers in forensic analysis laboratories, as well as researchers actively trying to sequence the genomes of unculturable cells. The present invention has use for tests such as DNA fingerprinting and mitochondrial typing. With the addition of blocker DNA, researchers and forensic analysts will be able to accurately amplify, PCR, and sequence samples that were previously limited by its quantity.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a graph illustrating Pico Green results of amplified K562 Human Genomic DNA and negative controls over time with and without cross-linked SS DNA with an initial starting mass of 1 pg.

FIG. 2 is a graph illustrating Pico Green results of amplified K562 Human Genomic DNA and negative controls over time with and without cross-linked SS DNA with an initial starting mass of 100 fg.

FIG. 3 is a graph illustrating Pico Green results of amplified K562 Human Genomic DNA and negative controls over time with and without cross-linked SS DNA with an initial starting mass of 10 fg.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Many challenges arise when trying to amplify and analyze human samples collected in the field due to limitations in sample quantity, and contamination of the starting material. Tests such as DNA fingerprinting and mitochondrial typing require a minimum template mass and are carried out in large volume reactions; in cases where insufficient sample is present whole genome amplification (WGA) can be used. WGA allows very small quantities of DNA to be amplified in a way that enables subsequent DNA-based tests to be performed. A limiting step to WGA is sample preparation. To minimize the necessary sample size, Applicants have developed a modification of WGA that allows for an increase in amplified product from small, nanoscale, purified samples with the use of carrier DNA. This technique addresses the limitations of sample size by providing ample copies of genomic samples. Carrier DNA, included in Applicants WGA reactions, can be used when amplifying samples with a standard purification method to potentially increase the amount of starting sample for future forensic DNA-based assays.

There are two major problems presented with regards to DNA purification and subsequent forensic analysis: 1) Forensic samples arrive in various physical forms such as hair, bone, blood, semen, vaginal fluids, skins cells, saliva, and tissue from which DNA must be isolated and purified. 2) The amount of DNA collected from the purification steps is always subject to a percent loss, and when the starting masses are small, the percent loss can be a significant proportion of template. Purified DNA is used for human typing and forensic analysis, which, depending on the assay and the amount of template collected, can exhaust the entire purified sample. Aside from limiting post clean-up analysis to a few forensic applications the percent loss can ultimately cause the purified sample to not satisfy the starting mass requirement for varying forensic techniques. Thus, the loss of purified DNA from clean-up can be compounded by the exhaustion of what little sample was available by a single forensic assay. Whole genome amplification is able to bypass the constraints of limited starting material on carrying out post-purification assays. However, WGA is generally thought of only if insufficient amounts of DNA are present for all desired forms of analysis. If sample size is not a limiting factor, there are a number of processes that are effective in the field of forensics.

When only smaller sample sizes are available for forensic analysis, WGA reactions are optimal. Increasing the chances of amplifying small samples can happen when the small amount of DNA is already purified, the efficiency with which the DNA is amplified can be improved by using carrier DNA.

### Cross-Linked Salmon Sperm DNA

Whole genome amplification (WGA) of DNA can currently be performed by utilizing kits, which contain phi29 polymerase and random priming hexamers. Phi29 polymerase is highly processive as well as accurate with its 3′-5′ Exonuclease activity making it an ideal candidate for whole genome amplification. Using it as a method for WGA is called Multiple Displacement Amplification (MDA) due to the displacement of newly replicated strands of DNA by the polymerase as it replicates complimentary strands. Multiple Displacement Amplification is defined as one cycle amplification of genomic DNA using exonuclease-resistant random primers and DNA polymerase with great processivity. Although effective when used with 1 ng of starting template or above, these kits are hindered by the amount of starting template needed to replicate multiple copies of DNA. The lower limit of templates makes it virtually impossible to amplify and sequence the genome of single cells as well as DNA templates less than 1 ng. A small quantity of template can also hinder the MDA reaction based on the template to reaction volume ratio. When a template is small in comparison to the reaction volume it is possible for the template to be passed over by the reagents.

Applicants have developed a method using cross-linked salmon sperm DNA as carrier DNA in conjunction with MDA that led to successful post-phi29 amplification of low mass samples. 83.3% of DNA samples in 10 mM Tris containing 13.6 fg of DNA, and 83.3% of samples in an STR mix containing 15.1 fg of DNA were successfully amplified post MDA with mitochondrial primers. However, of the samples not containing cross-linked salmon sperm DNA 50.0% of samples in 10 mM Tris containing 13.6 fg of DNA and 33.3% of samples in STR mix containing 15.1 fg of DNA were positively amplified post MDA with mitochondrial primers. The products from MDA with cross-linked carrier DNA were successfully amplified with mitochondrial primers in a PCR reaction, as well as sequenced without major error found between the sequence of the unamplified sample vs. the sequence of the amplified sample. Thus, the addition of cross-linked DNA can aid in attempts to sequence unculturable prokaryotic and eukaryotic cells, as well as help forensic scientists positively or negatively identify suspects in crime cases with low quantities of DNA evidence.

Applicants used cross-linked salmon sperm DNA as carrier in one example as follows: DNA 10 μl of stock salmon sperm DNA (10 mg/ml, Invitrogen, Carlsbad, Calif.) was diluted in 90 μl of 10 mMTris (pH 8.0). The tube was vortexed briefly and then cross-linked in a UV Stratalinker 1800 (Stratagene, La Jolla, Calif.) for 10 minutes at 254 nanometers, briefly vortexed and cross-linked again for an additional 10 minutes. The contents were transferred to a 1.5 ml microfuge tube for an ethanol precipitation. 200 μl chilled (−20° C.) 100% ethanol was added, mixed, and then 2 μl of cold glycogen 20 mg/ml (Roche, Indianapolis, Ind.) was added. The tube was stored at −80° C. overnight. In the morning the tube was spun at 14,000 rpm's on an Eppendorf 5804R centrifuge for 30 minutes at 4° C. The supernatant was poured, then pipetted off and the pellet was allowed to air dry. The pellet was then resuspended in 1-ml of 10 mM Tris (pH 8.0) and stored at 4° C. overnight. The treated salmon sperm DNA was later diluted down to 100 pg/μL with 10 mM Tris (pH 8.0).

Whole genome amplification was performed by mixing 5 μl of 2 mM dNTP (Roche, Indianapolis, Ind.), 4 μl of 1×phi29 Buffer mix(10 μl of 10× Buffer, 2 μl of 10 mg/ml BSA, 88 μl of dH₂0, NEB, Beverly, Mass.), 1 μl of 4 mM DTT (Molecular Staging, New Haven, Conn.), and 1 μl of 2 mM Random Hexamers 5′-nnnnnn-3′, where n=C,T,G, or A (Genosys, Woodlands, Tex.). The reagents were mixed and 1 μl of 100 pg/μl UV'd Salmon Sperm DNA was added. The contents were incubated at room temperature for 10 minutes, after which point 5 μl of K562 human genomic DNA (Promega, Madison, Wis.) sample was added and mixed. The tube was transferred to a DNA Engine PTC-200 Thermocycler (MJR Research, Waltham, Mass.) where it was heat denatured at 95° C. for 1 minute, lowered to 25° C. for 3 minutes for the addition of 0.5 μl of Phi29 Polymerase (10 U/μl, NEB), incubated at 30° C. for 16 hours, the polymerase was heat denatured at 65° C. for 10 minutes, and lowered to 4° C. until they could be moved to the −20° C. freezer for future mitochondrial PCR amplification and sequencing.

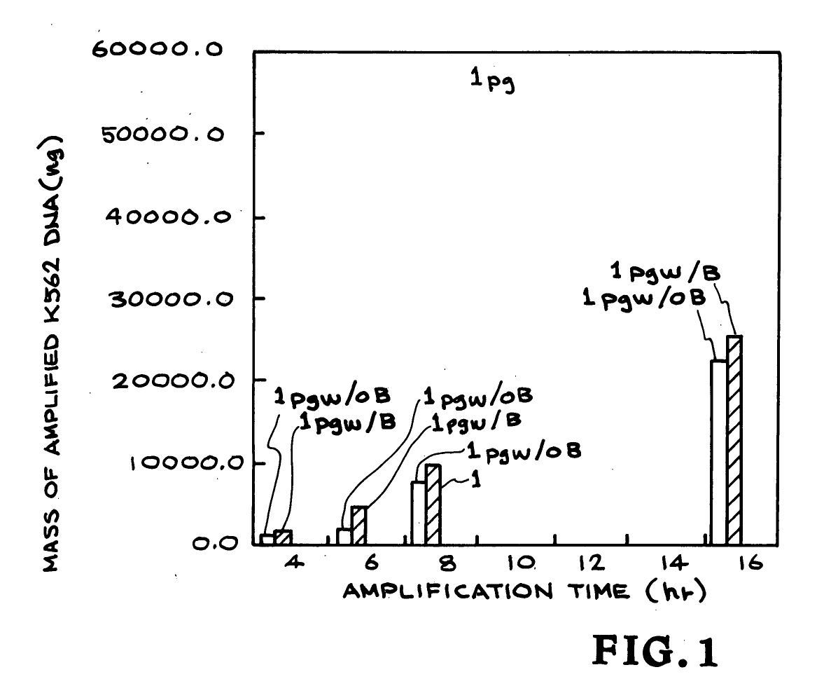

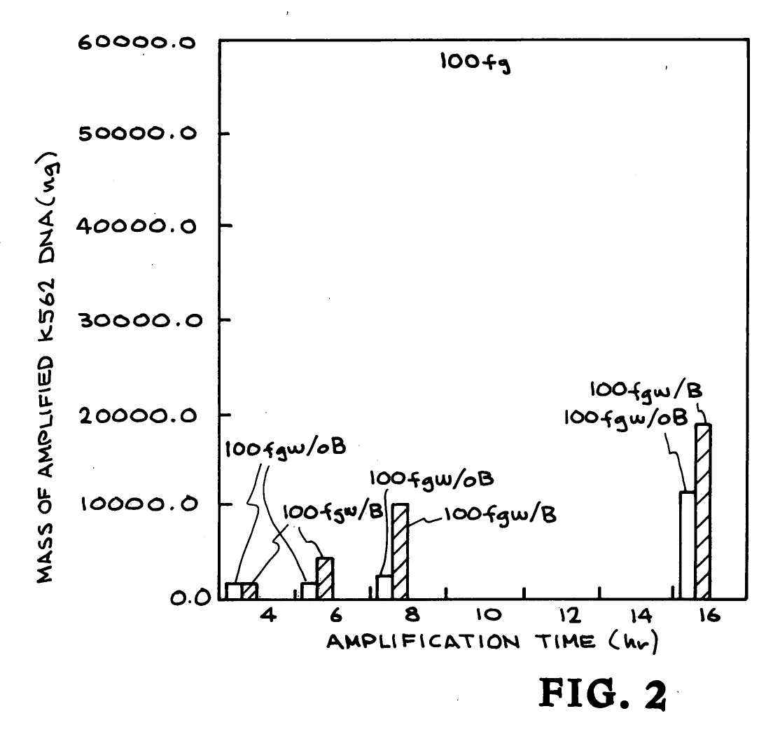

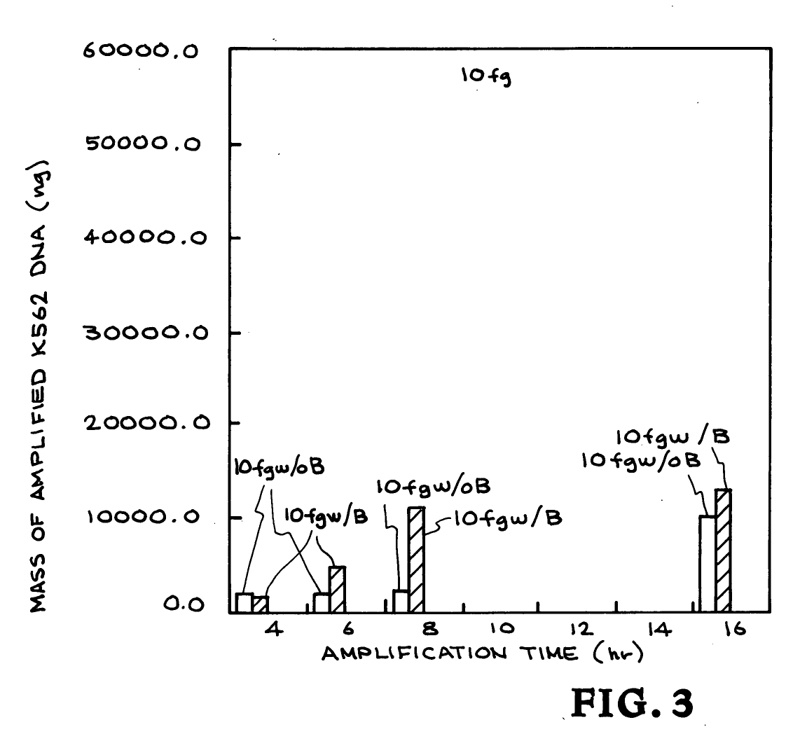

Referring to the drawings, Pico Green results of amplified K562 Human Genomic DNA and negative controls over time with and without cross-linked SS DNA are illustrated. Referring to FIG. 1, a graph illustrates Pico Green results of amplified K562 Human Genomic DNA and negative controls over time with and without cross-linked SS DNA with an initial starting mass of 1 pg. Referring to FIG. 2 a graph illustrates Pico Green results of amplified K562 Human Genomic DNA and negative controls over time with and without cross-linked SS DNA with an initial starting mass of 100 fg. Referring to FIG. 3, a graph illustrates Pico Green results of amplified K562 Human Genomic DNA and negative controls over time with and without cross-linked SS DNA with an initial starting mass of 10 fg.

Methodology of Small Sample WGA with blocker DNA Carrier DNA

Carrier DNA used in this study was Salmon Sperm DNA (Invitrogen,) that was prepared by cross-linking under UV light with a subsequent ethanol precipitation.

Sample preparation:

The DNA amplified in this experiment was double stranded human genomic DNA K562 (Promega). One set of samples was diluted in 10 mM Tris, pH 8.0, while a second set was diluted in an STR PCR mixture (Promega), excluding the polymerase. These two sample conditions were chosen based on the potential condition that forensic samples may undergo MDA in a casework scenario. Sample set one was composed of 100 ng, 50 ng, 10 ng, 1.0 ng, 100 pg, 50 pg, 10 pg, and 1 pg of DNA; each diluted in 10 mM Tris to a final volume of 20 μl. The same amounts of DNA were diluted in the STR PCR mix supplied with the GenePrint Fluorescent STR Multiplex-GammaSTR (Promega). The STR PCR mix for each DNA sample was brought to a final volume of 22.3 μl with dH20.

Whole Genome Amplification using Multiple Displacement Amplification with and without salmon sperm DNA-WGA with phi29 polymerase, random priming hexamers and cross-linked salmon sperm DNA was performed in a thin-walled 0.2 ml microfuge tube in a total volume following a modified protocol. 11 ul's of mastermix were aliquoted into each tube, at which point the cross-linked Salmon Sperm DNA (Invitrogen) was added and briefly incubated. DNA samples in either Tris or STR mix were added to each tube and then heat denatured at 95° C. for 1 minute, quickly lowered to 25° C. for the addition of phi29 polymerase, incubated at 30° C. for 16 hours, heat-denatured at 65° C. for 10 minutes and then maintained at 4° C. until post-amplification applications could be ran.

WGA with phi29 polymerase but without the addition of cross-linked Salmon Sperm DNA was also performed on the prepared DNA samples. The reaction mastermix was prepared as above but without the addition of cross-linked Salmon Sperm DNA. The samples were subjected to the same thermal profile and storage conditions as those containing cross-linked salmon sperm DNA.

Mitochondrial PCR analysis

Mitochondrial PCR (mtPCR) was used to visualize the success rate of cross-linked Salmon Sperm DNA. The regions HV1, HV2 and HV1B from mitochondrial DNA (mtDNA) were Applicants targets.

Pico Green Analysis

Pico-green double stranded DNA (dsDNA) quantitation reagent (Molecular Probes) was employed to quantify the relationship between amplification and the addition of cross-linked salmon sperm DNA. Reagent preparation was performed according to the protocol provided with the kit. A five point high range standard curve and a seven point low range standard curve were prepared from Lambda DNA to estimate the concentration of DNA in each sample. Three separate dilutions of each initial DNA concentration sample were made in order to obtain an average fluorescence value. To each tube, 1.0 ml of the working solution of PicoGreen reagent was added. The fluorescence of the DNA samples was measured with the instrument calibrated to the low standard curve first. Any samples that had higher fluorescence intensity than the fluorometer's maximum for the low standard curve were measured again after the instrument was calibrated to the high standard curve. The fluorescence value of the reagent blank (1× TE) from the corresponding:

Small sample size is a pertinent issue that needs to be addressed in Forensic science.

Small samples initially amplified with WGA and cross-linked salmon sperm DNA were amplified more successfully with mitochondrial DNA PCR than the samples lacking the cross-linked salmon sperm DNA.

Pico Green analysis supports the results found from the mitochondrial DNA PCR, in that the addition of cross linked salmon sperm DNA results in a greater yield of amplified DNA.

The negative controls, both with and without the cross-linked salmon sperm DNA, did not yield a significant amount of amplified DNA that would negatively influence other post amplification analysis.

A combination of phi 29 polymerase, random priming hexamers and cross-linked salmon sperm DNA make a more robust assay than what is currently available.

The results of this study demonstrate that MDA with the addition of cross-linked salmon sperm DNA can enable a lower detection limit for mtDNA PCR than similar samples subjected to MDA without cross-linked Salmon sperm DNA.

Downstream forensic applications can be applied even when starting material is as low as the picogram amount or possibly even lower yet.

Applicants have conducted research and analysis of the present invention and the following article describes some of that research and analysis: “Small sample whole-genome amplification,” by Christine Hara, Christine Nguyen, Elizabeth Wheeler, Karen Sorensen, Erin Arroyo, Greg Vrankovich, and Allen Christian, Proc. SPIE 6007, 600717 (2005). The article, “Small sample whole-genome amplification,” by Christine Hara, Christine Nguyen, Elizabeth Wheeler, Karen Sorensen, Erin Arroyo, Greg Vrankovich, and Allen Christian, Proc. SPIE 6007, 600717 (2005), is incorporated herein by this reference.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is

1. A method of amplifying a nucleic acid sample, comprising the steps of:
bringing the nucleic acid sample together with blocking DNA,
adding DNA polymerase, and
performing multiple displacement amplification.

2. The method of amplifying a nucleic acid sample of claim 1 wherein the method is adapted to amplify nucleic acid of a specific species of organism and said step of bringing the nucleic acid sample together with blocking DNA comprises bringing the nucleic acid sample together with blocking DNA from a species other than said specific species of organism.

3. The method of amplifying a nucleic acid sample of claim 1 wherein the method is adapted to amplify nucleic acid of a human species and said step of bringing the nucleic acid sample together with blocking DNA comprises bringing the nucleic acid sample together with blocking DNA from a species other than said human species.

4. The method of amplifying a nucleic acid sample of claim 1 wherein said step of bringing the nucleic acid sample together with blocking DNA comprises bringing the nucleic acid sample together with cross-linked salmon sperm DNA.

5. The method of amplifying a nucleic acid sample of claim 1 including performing polymerase chain reaction for further amplification.

6. A method of amplifying a nucleic acid sample, comprising the steps of:
providing a starting template of the nucleic acid sample,
bringing said template together with blocking DNA,
bringing said template together with DNA polymerase, and
multiple displacement amplification of said template.

7. The method of amplifying a nucleic acid sample of claim 6 wherein said step of providing a starting template comprises providing a starting template of less than 1 ng of the nucleic acid sample.

8. The method of amplifying a nucleic acid sample of claim 6 wherein the method is adapted to amplify nucleic acid of a specific species of organism and said step of bringing said template together with blocking DNA comprises bringing said template together with blocking DNA from a species other than said specific species of organism.

9. The method of amplifying a nucleic acid sample of claim 6 wherein the method is adapted to amplify nucleic acid of a human species and said step of bringing said template together with blocking DNA comprises bringing said template together with blocking DNA from a species other than said human specific species.

10. The method of amplifying a nucleic acid sample of claim 6 wherein said step of bringing said template together with blocking DNA, comprises bringing said template together with cross-linked salmon sperm DNA.

11. The method of amplifying a nucleic acid sample of claim 6 including performing polymerase chain reaction of said template for further amplification.

12. A method of whole genome amplification of a nucleic acid sample, comprising the steps of:
providing a starting template of the nucleic acid sample,
bringing said template together with blocking DNA,
bringing said template together with DNA polymerase, and
multiple displacement amplification of said template.

13. The method of whole genome amplification of a nucleic acid sample of claim 12 wherein said step of providing a starting template comprises providing a starting template of less than 1 ng of the nucleic acid sample.

14. The method of whole genome amplification of a nucleic acid sample of claim 12 wherein the method is adapted to amplify nucleic acid of a specific species of organism and said step of bringing said template together with blocking DNA comprises bringing said template together with blocking DNA from a species other than said specific species of organism.

15. The method of whole genome amplification of a nucleic acid sample of claim 12 wherein the method is adapted to amplify nucleic acid of a human species and said step of bringing said template together with blocking DNA comprises bringing said template together with blocking DNA from a species other than said human specific species.

16. The method of whole genome amplification of a nucleic acid sample of claim 12 wherein said step of bringing said template together with blocking DNA, comprises bringing said template together with cross-linked salmon sperm DNA.

17. The method of whole genome amplification of a nucleic acid sample of claim 12 including performing polymerase chain reaction of said template for further amplification.
